(19) United States
(12) Patent Application Publication
(10) Pub. No.: US 2006/0264672 A1
(43) Pub. Date: Nov. 23, 2006

(54) Processes using alpha, omega-difunctional aldaramides as monomers and crosslinkers

(76) Inventors: Mark Allen Andrews, Wilmington, DE (US); Henry Keith Chenault, Hockessin, DE (US); Garret D. Figuly, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/360,308

(22) Filed: Feb. 23, 2006

Related U.S. Application Data
(60) Provisional application No. 60/655,282, filed on Feb. 23, 2005.

(51) Int. Cl. C07C 237/00

(52) U.S. Cl. 564/152

(57) ABSTRACT

Processes using alpha, omega-difunctional aldaramides as monomers and crosslinkers are disclosed. The processes can be used to form polymers, particularly crosslinked polymers.

### FIELD OF INVENTION

The invention is directed to processes using alpha, omega-difunctional aldaramides as monomers and crosslinkers.

### BACKGROUND

The concept of using biomass-derived materials to produce other useful products has been explored since man first used plant materials and animal fur to make clothing and tools. Biomass derived materials have also been used for centuries as adhesives, solvents, lighting materials, fuels, inks/paints/coatings, colorants, perfumes and medicines. Recently, people have begun to explore the possibility of using “refined biomass” as starting materials for chemical conversions leading to novel high value-in-use products. Over the past two decades, the cost of renewable biomass materials has decreased to a point where many are competitive with those derived from petroleum. In addition, many materials that cannot be produced simply from petroleum feedstocks are potentially available from biomass or refined biomass. Many of these unique, highly functionalized, molecules would be expected to yield products unlike any produced by current chemical processes. “Refined biomass” is purified chemical compounds derived from the first or second round of plant biomass processing. Examples of such materials include cellulose, sucrose, glucose, fructose, sorbitol, erythritol, and various vegetable oils.

A particularly useful class of refined biomass is that of aldaric acids. Aldaric acids, also known as saccharic acids, are diacids derived from naturally occurring sugars. When aldoses are exposed to strong oxidizing agents, such as nitric acid, both the aldehydic carbon atom and the carbon bearing the primary hydroxyl group are oxidized to carboxyl groups. An attractive feature of these aldaric acids includes the use of very inexpensive sugar based feedstocks, which provide low raw material costs and ultimately could provide low polymer costs if the proper oxidation processes are found. Also, the high functional density of these aldaric acids provide unique, high value opportunities, which are completely unattainable at a reasonable cost from petroleum based feedstocks.

Aldaric acid derivatives, because of their high functionality, are potentially valuable monomers and crosslinking agents. Co-pending patent application Ser. Nos. 11/064,191 and 11/064,192 describe the use of some of these materials in the preparation of cross-linked polymers.

Diaminoaldaramides, dihydroxyaldaramides, bis(alkoxycarbonylalkyl)aldaramides, and bis(carboxyalkyl)aldaramides are examples of monomers and crosslinking agents that could be prepared. Co-pending patent application 60/655,647 describes the preparation of some of these types of compounds. U.S. Pat. No. 5,496,545 discloses crosslinked polyallylamine and polyethyleneimine. The crosslinking agents disclosed include epichlorohydrin, diepoxides, diisocyanates, α,ω-dihaloalkanes, diacrylates, bisacrylamides, succinyl chloride, and dimethyl succinate.

Applicants have invented a process to prepare new polymers and new crosslinked polymers, using monomers crosslinking moieties that could be derived from biomass sources.

### SUMMARY OF THE INVENTION

An aspect of the invention is a method of preparing a polymer comprising: contacting one or more suitable monomers with a compound of Formula I, V or XXII:

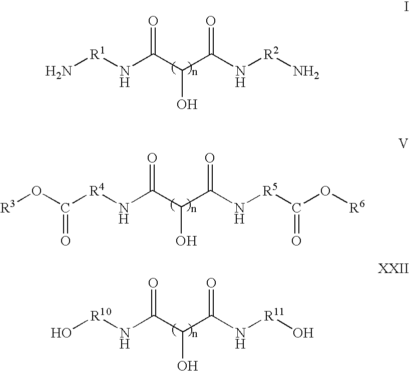

wherein n=1-6, R¹, R², R⁴, R⁵, R¹⁰, and R¹¹ are independently optionally substituted hydrocarbylene groups, wherein the hydrocarbylene groups are aliphatic or aromatic, linear, branched, or cyclic, and wherein the hydrocarbylene groups optionally contain —O— linkages, and R³ and R⁶ are independently hydrogen, optionally substituted aryl or optionally substituted alkyl.

In some embodiments of the invention, the compounds of Formula 1, V or XXII are prepared in situ.

Another aspect of the invention is a polymer made by the method of described above.

Another aspect of the invention is a method to crosslink a polymer comprising contacting a suitable polymer with one or more crosslinking agents of Formula I, V or XXII:

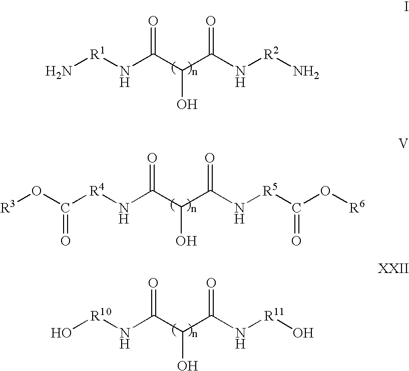

wherein n=1-6, R¹, R², R⁴, R⁵, R¹⁰, and R¹¹ are independently optionally substituted hydrocarbylene groups, wherein the hydrocarbylene groups are aliphatic or aromatic, linear, branched, or cyclic, and wherein the hydrocarbylene groups optionally contain —O— linkages, and R³ and R⁶ are independently hydrogen, optionally substituted aryl or optionally substituted alkyl.

Another aspect of the invention is a polymer made by a method comprising: contacting one or more suitable monomers with a compound of Formula I, V or XXII:

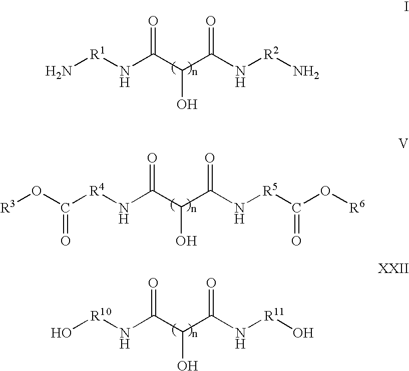

wherein n=1-6, R¹, R², R⁴, R⁵, R¹⁰, and R¹¹ are independently optionally substituted hydrocarbylene groups, wherein the hydrocarbylene groups are aliphatic or aromatic, linear, branched, or cyclic, and wherein the hydrocarbylene groups optionally contain —O— linkages, and R³ and R⁶ are independently hydrogen, optionally substituted aryl or optionally substituted alkyl.

These and other aspects of the invention will be apparent to those skilled in the art in view of the following description and the appended claims.

### DETAILED DESCRIPTION

The following definitions may be used for the interpretation of the present specification and the claims:

By hydrocarbyl is meant a straight chain, branched or cyclic arrangement of carbon atoms connected by single, double, or triple carbon-to-carbon bonds, and substituted accordingly with hydrogen atoms. Hydrocarbyl groups can be aliphatic and/or aromatic. Examples of hydrocarbyl groups include methyl, ethyl, propyl, isopropyl, butyl, isobutyl, t-butyl, cyclopropyl, cyclobutyl, cyclopentyl, methylcyclopentyl, cyclohexyl, methylcyclohexyl, benzyl, phenyl, o-tolyl, m-tolyl, p-tolyl, xylyl, vinyl, allyl, butenyl, cyclohexenyl, cyclooctenyl, cyclooctadienyl, and butynyl. Examples of substituted hydrocarbyl groups include toluyl, chlorobenzyl, —(CH₂)—O—(CH₂)—, fluoroethyl, p-(CH₃S)C₆H₅, 2-methoxypropyl, and (CH₃)₃SiCH₂.

“Alkyl” means a saturated hydrocarbyl group. Examples of alkyl groups include methyl, ethyl, propyl, isopropyl, butyl, s-butyl, isobutyl, pentyl, neopentyl, hexyl, heptyl, isoheptyl, 2-ethylhexyl, cyclohexyl and octyl.

“Aryl” means a group defined as a monovalent radical formed conceptually by removal of a hydrogen atom from a hydrocarbon that is structurally composed entirely of one or more benzene rings. Examples of aryl groups include benzene, biphenyl, terphenyl, naphthalene, phenyl naphthalene, and naphthylbenzene.

‘Alkylene’ and ‘arylene’ refer to the divalent forms of the corresponding alkyl and aryl groups. ‘Hydrocarbylene’ groups include ‘alkylene’ groups, ‘arylene’ groups, and groups that can be represented by connecting some combination of alkylene and arylene groups. “Divalent”, as used herein, means that the groups can form two bonds.

“Substituted” and “substituent” mean containing one or more substituent groups, or “substituents,” that do not cause the compound to be unstable or unsuitable for the use or reaction intended. Unless otherwise specified herein, when a group is stated to be “substituted” or “optionally substituted”, substituent groups that can be present include carboxyl, carboxamide, nitrile, ether, ester, halogen, amine (including primary, secondary and tertiary amine), hydroxyl, oxo, imine, oxime, silyl, siloxy, nitro, nitroso, thioether, sulfoxide, sulfone, sulfonate ester, sulfonamide, sulfonic acid, phosphine, phosphoryl, phosphonyl, phosphonamide, and salts thereof.

The present invention is directed to methods of preparing polymers using difunctional aldaramides as monomers, and to methods of crosslinking polymers using difunctional aldaramides as crosslinkers. Co-pending patent applications Ser. Nos. 11/064,191 and 11/064,192 herein incorporated entirely by reference, describe the use of some of these materials in the preparation of cross-linked polymers. Co-pending patent application 60/655,647, herein incorporated entirely by reference, describes the preparation of some of these difunctional aldaramides.

Aldaric acids are diacids derived from naturally occurring sugars. When aldoses are exposed to strong oxidizing agents, such as nitric acid, both the aldehydic carbon atom and the carbon bearing the primary hydroxyl group are oxidized to carboxyl groups. This family of diacids is known as aldaric acids (or saccharic acids). An aldarolactone has one carboxylic acid lactonized; the aldarodilactone has both lactonized. As illustration, the aldaric acid derivatives starting from D-glucose are shown below.

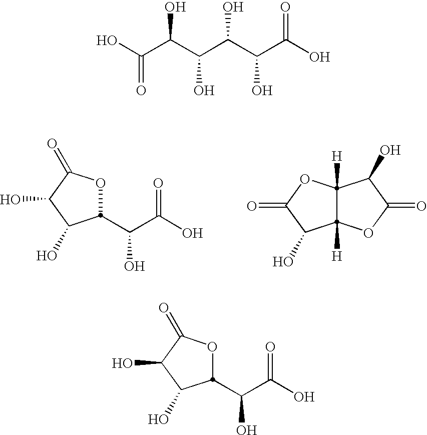

The compounds used in the processes disclosed herein and their starting materials can be made from aldaric acids or their derivatives, or from any other source. Any stereoisomer or mixture of stereoisomers can be used in the compositions and processes disclosed herein.

One aspect of the invention is a method of preparing a polymer comprising: contacting one or more suitable monomers with a compound of Formula I, V or XXII.

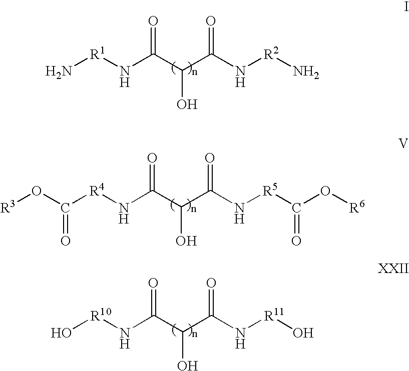

wherein n=1-6, R¹, R², R⁴, R⁵, R¹⁰, and R¹¹ are independently optionally substituted hydrocarbylene groups, wherein the hydrocarbylene groups are aliphatic or aromatic, linear, branched, or cyclic, and wherein the hydrocarbylene groups optionally contain —O— linkages, and R³ and R⁶ are independently hydrogen, optionally substituted aryl or optionally substituted alkyl.

In some embodiments of the invention, n is equal to 4.

R¹, R², R⁴, R⁵, R¹⁰, and R¹¹ can be independently alkylene, polyoxaalkylene, or arylene groups, linear or branched, wherein the alkylene, polyoxaalkylene, or arylene groups are optionally substituted with NH₂ or alkyl. When R¹, R², R¹⁰, or R¹¹ is alkylene, it can have from 2 to 20 carbon atoms, preferably from 2 to 8. When R⁴ or R⁵ is alkylene, it can have from 1 to 12 carbon atoms, preferably from 1 to 6. In some embodiments, R¹ and R², R⁴ and R⁵, R³ and R⁶, or R¹⁰ and R¹¹ can be the same.

By “polyoxaalkylene” is meant linear or branched alkyl groups linked by ether linkages. Polyoxaalkylene can contain 2 carbons up to polymeric length units. Examples of polymeric polyoxaalkylenes suitable for the present inventions include polyethyleneglycols, polypropylene glycols, and polytetramethylene glycols such as those based on Terathane® polytetramethyleneetherglycol (E. I. DuPont de Nemours, Wilmington, Del.).

In some embodiments, R¹, R², R⁴, R⁵, R¹⁰, and/or R¹¹ can be an alkylene, polyoxaalkylene, heteroarylene, or arylene group, linear or branched, wherein the alkylene, polyoxaalkylene, heteroarylene or arylene group is optionally substituted with NH₂, aryl including heteroaryl, or alkyl. In some embodiments, n is 4. When R¹, R², R¹⁰, or R¹¹ is alkylene, it can have from 2 to 20 carbon atoms, preferably from 2 to 8. When R⁴ or R⁵ is alkylene, it can have from 1 to 12 carbon atoms, preferably from 1 to 6. Also, “arylene” is intended to include arenedialkylene, e.g.,

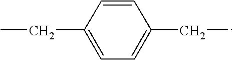

When R¹, R², R⁴, R⁵, R¹⁰, and/or R¹¹ is arylene, it can have from 2 to 12 carbon atoms, preferably 4 to 6. For example, when R¹, R², R⁴, R⁵, R¹⁰, and/or R¹¹ has two carbon atoms, it can be a heteroarylene, e.g., a triazole ring. When R¹, R², R⁴, R⁵, R¹⁰, and/or R¹¹ has 12 carbon atoms, it can be, for example, a biphenyl. When R¹, R², R⁴, R⁵, R¹⁰, and/or R¹¹ has 4 carbon atoms, examples are furan or pyrrole rings.

When R¹, R², R⁴, R⁵, R¹⁰, and/or R¹¹ is polyoxaalkylene, it can have from 1 to 50 repeat units, preferably from 1 to 10. The total number of carbons depends on the number of carbons in the repeat unit.

For a compound of Formula I, suitable monomers are monomers that react with the primary amine groups of Formula I at a temperature less than or equal to about 130° C. to form carbon-nitrogen bonds. Such compounds include bis(acyl chlorides), bis(acyl bromides), bis(acyl iodides), bis(carboxylic acid anhydrides), diesters, alkyl dichlorides, alkyl dibromides, alkyl diiodides, alkyl bis(sulfonate esters), diepoxyalkanes, diisocyanates, carbonate esters, phosgene, carbonyldiimidazole, epichlorohydrin and dicarboxylic acids in combination with a dehydrating agent that promotes amide bond formation between the primary amine groups of compounds of Formula I and the carboxyl groups of the dicarboxylic acid. It is understood that some of these species can be interchanged or generated in situ. For example, an acyl or alkyl chloride can be converted in situ to a more reactive acyl or alkyl bromide or iodide by reaction with a bromide or iodide salt, such as sodium or potassium bromide, sodium or potassium iodide or a tetraalkylammonium bromide or iodide, such as tetrabutylammonium bromide or iodide. A carboxylic acid can be converted in situ into a mixed anhydride by reaction with isobutyl chloroformate. Examples of bis(acyl chlorides) include succinyl dichloride, glutaryl dichloride, adipoyl dichloride, suberoyl dichloride, sebacoyl dichloride, isophthaloyl dichloride, terephthaloyl dichloride, 4,4′-oxybisbenzoyl chloride, 3,3′-methylenebisbenzoyl chloride, bicyclo[2.2.1]hept-5-ene-2,3-dicarbonyl dichloride, 2,3,4,5-tetraacetoxyadipoyl dichloride, 3,6,9-trioxaundecane-1,11-dioic chloride, 3,6-dioxaoctane-1,8-dioic chloride, 3-oxapentane-1,5-dioic chloride, and polyethylene glycol bis(chloroformylmethyl)ether. Examples of bis(acyl bromides) include succinyl dibromide, glutaryl dibromide, adipoyl dibromide, suberoyl dibromide, sebacoyl dibromide, isophthaloyl dibromide, terephthaloyl dibromide, 4,4′-oxybisbenzoyl bromide, 3,3′-methylenebisbenzoyl bromide, and bicyclo[2.2.1]hept-5-ene-2,3-dicarbonyl dibromide. Examples of bis(acyl iodides) include succinyl diiodide, glutaryl diiodide, adipoyl diiodide, suberoyl diiodide, sebacoyl dibromide, isophthaloyl diiodide, terephthaloyl diiodide, 4,4′-oxybisbenzoyl iodide, 3,3′-methylenebisbenzoyl iodide, and bicyclo[2.2.1]hept-5-ene-2,3-dicarbonyl diiodide. Examples of bis(carboxylic acid anhydrides) include sebacic bis(trichloroacetic) anhydride, adipoyl bis(isobutylcarbonate), and 1,2,4,5-benzenetetracarboxylic acid dianhydride. Diesters may have any of a number of reactive ester groups, including methyl, ethyl, 2,2,2-trifluoroethyl, N-succinimidyl, 1-benzotriazolyl, phenyl, pentafluorophenyl, 4-nitrophenyl ester groups. Examples of diesters include bis(2,2,2-trifluoroethyl)succinate, bis(1-benzotriazolyl)glutarate, bis(pentafluorophenyl) adipate, dimethyl suberate, diethyl sebacate, bis (2,2,2-trifluoroethyl)isophthalate, bis(4-nitrophenyl) terephthalate, bis(1-benzotriazolyl)4,4′-oxydibenzoate, dimethyl 4,4′-methylenedibenzoate, and polyethylene glycol bis(N-succinimidyloxycarbonylmethyl)ether.

Examples of alkyl dichlorides include 1,4-dichlorobutane, 1,5-dichloropentane, 1,6-dichlorohexane, 1,8-dichlorooctane, 1,8-dichloro-3,6-dioxaoctane, 1,11-dichloro-3,6,9-trioxaundecane, α,α′-dichloro-p-xylene, and α,α′-dichloro-m-xylene. Examples of alkyl dibromides include 1,4-dibromobutane, 1,5-dibromopentane, 1,6-dibromohexane, 1,8-dibromooctane, 1,8-dibromo-3,6-dioxaoctane, 1,11-dibromo-3,6,9-trioxaundecane, and α,α′-dibromo-p-xylene. Examples of alkyl diiodides include 1,4-diiodobutane, 1,5-diiodopentane, 1,6-diiodohexane, 1,8-diiodooctane, 1,8-diiodo-3,6-dioxaoctane, 1,11-diiodo-3,6,9-trioxaundecane, and α,α′-diiodo-p-xylene. Examples of sulfonate esters include methanesulfonate, p-toluenesulfonate, trifluoromethanesulfonate and 2,2,2-trifluoroethylsulfonate esters. Examples of alkyl bis(sulfonate esters) include 1,3-bis(p-toluenesulfonyloxy)propane, 1,4-bis(2,2,2-trifluoroethanesulfonyloxy)butane, 1,5-bis(trifluoromethanesulfonyloxy)pentane, 1,6-bis(methanesulfonyloxy)hexane, 1,8-bis(p-toluenesulfonyloxy)octane, 1,10-bis(trifluoromethanesulfonyloxy)decane, 1,12-bis(methanesulfonyloxy)dodecane, 1,14-bis(p-toluenesulfonyloxy)tetradecane, 1,16-bis(methanesulfonyloxy)hexadecane, 1,4-bis(methanesulfonyloxymethyl)benzene, 1,3-bis(p-toluenesulfonyloxymethyl)benzene, mannitol 1,6-dimethanesulfonate, 1,14-bis(trifluoromethanesulfonyloxy)-3,6,9,12-tetraoxatetradecane, 1,11-bis(trifluoromethanesulfonyloxy)-3,6,9-trioxaundecane, 1,8-bis(trifluoromethanesulfonyloxy)-3,6-dioxaoctane, polyethyleneglycolbis(methanesulfonate), and polyethylene glycol bis(p-toluenesulfonate). Examples of diepoxyalkanes include 1,3-diglycidyloxybenzene, 1,4-diglycidyloxybenzene, 1,2-diglycidyloxyethane, 1,4-bis(glycidyloxy)butane, 1,6-bis(glycidyloxy)hexane, 1,2:15,16-diepoxy-4,7,10,13-tetraoxahexadecane, 1,2:12,13-diepoxy-4,7,10-trioxatridecane, bis(4-glycidyloxyphenyl)methane, 1,2:7,8-diepoxyoctane, and 4,4′-diglycidyloxybiphenyl. Examples of diisocyanates include 1,4-diisocyanatobenzene, 1,3-diisocyanatobenzene, 2,6-diisocyanatotoluene, 4,4′-bis(isocyanatophenyl)methane, 1,4-diisocyanatocyclohexane, 1,3-diisocyanatocyclohexane, 1,3-bis(isocyanatomethyl)benzene, isophorone diisocyanate, 4,4′-diisocyanatodicyclohexylmethane, tetramethylene diisocyanate, hexamethylene diisocyanate, bis(2-isocyanatoethyl)ether, and 1,8-diisocyanato-3,6-dioxaoctane. Examples of carbonate esters include dimethyl carbonate, diethyl carbonate, dipropyl carbonate, diphenyl carbonate, bis(trichloromethyl)carbonate, and bis(pentafluorophenyl)carbonate. Dicarboxylic acids include succinic acid, glutaric acid, adipic acid, suberic acid, sebacic acid, dodecanedioic acid, tetradecanedioic acid, hexadecanedioic acid, terephthalic acid, isophthalic acid, 1,3-cyclohexanedicarboxylic acid, 1,4-cyclohexanedicarboxylic acid, 4,4′-biphenyldicarboxylic acid, 4,4′-oxybis(benzoic acid), 4,4′-methylenebis(benzoic acid), 3,3′-oxybis(benzoic acid), 3,3′-methylenebis(benzoic acid), 3,6,9-trioxaundecane-1,11-dioic acid, 3,6-dioxaoctane-1,8-dioic acid, 3-oxapentane-1,5-dioic acid, and polyethylene glycol bis(carboxymethyl)ether. Dehydrating agents that promote amide bond formation between the primary amine groups of compounds of Formula I and the carboxyl groups of the dicarboxylic acids include carbodiimides, such as dicyclohexylcarbodiimide and 1-ethyl-3-(3-dimethylaminopropyl)carbodiimide hydrochloride, and benzotriazol-1-yloxy reagents, such as 2-(1H-benzotriazol-1-yl)-1,1,3,3-tetramethyluronium hexafluorophosphate (HBTU) and benzotriazol-1-yloxytris(dimethylamino)phosphonium hexafluorophosphate (BOP).

For a compound of Formula V, suitable monomers are monomers that react with the ester groups of Formula V at a temperature less than or equal to about 130° C. to form bonds to the carbonyl carbon atoms. Such suitable monomers include diamines and dithiols. Examples of diamines include tetramethylenediamine, pentamethylenediamine, hexamethylenediamine, heptamethylenediamine, octamethylenediamine, nonamethylenediamine, decamethylenediamine, undecamethylenediamine, diethylenetriamine, triethylenetetramine, tetraethylenepentamine, pentaethylenehexamine, 4-aza-1,7-diaminoheptane, spermine, spermidine, 1,9-diamino-3,7-diazanonane, 1,11-diamino-4,8-diazaundecane, 1,10-diamino-4,7-diazadecane, bis(hexamethylene)triamine, 1,4-bis(aminomethyl)benzene, 1,3-bis(aminomethyl)benzene, 1,4-bis(2-aminoethyl)benzene, 1,3-bis(2-aminoethyl)benzene, 1,5-diamino-3-thiapentane, 1,5-diamino-3-oxapentane, 1,8-diamino-3,6-dioxaoctane, 1,11-diamino-3,6,9-trioxaundecane, 1,7-diamino-4-oxaheptane, 1,10-diamino-4,7-dioxadecane, 1,13-diamino-4,7,10-trioxatridecane, 1,12-diamino-4,9-dioxadodecane, 4,4′-bis(aminomethyl)biphenyl, 1,4-bis(aminomethyl)cyclohexane, 1,3-bis(aminomethyl)cyclohexane, 4,4′-oxybisbenzylamine, 3,3′-oxybisbenzylamine, 4,4′-methylenebisbenzylamine, 3,3′-methylenebisbenzylamine, polyethylene glycol bis(2-aminoethyl)ether, isophorone diamine and dilysine. Examples of dithiols include 1,4-butanenedithiol, 1,5-pentanenedithiol, 1,6-hexanenedithiol, 1,7-heptanenedithiol, 1,8-octanenedithiol, 1,9-nonanenedithiol, 1,10-decanenedithiol, 1,4-bis(thiomethyl)benzene, 1,3-bis(thiomethyl)benzene, 3-thiapentane-1,5-dithiol, 3-oxapentane-1,5-dithiol, 3,6-dioxaoctane-1,8-dithiol, trioxaundecane-1,11-dithiol, and polyethylene glycol bis(2-thioethyl)ether.

For a compound of Formula XXII, suitable monomers are monomers that react with the hydroxyl groups of Formula XXII at a temperature less than or equal to about 130° C. to form carbon-oxygen bonds. Such compounds include bis(acyl chlorides), bis(acyl bromides), bis(acyl iodides), alkyl dichlorides, alkyl dibromides, alkyl diiodides, alkyl bis(sulfonate esters), diepoxyalkanes, diisocyanates, phosgene, carbonyldiimidazole and epichlorohydrin. It is understood that some of these species can be interchanged or generated in situ. For example, an acyl or alkyl chloride can be converted in situ to a more reactive acyl or alkyl bromide or iodide by reaction with a bromide or iodide salt, such as sodium or potassium bromide, sodium or potassium iodide or a tetraalkylammonium bromide or iodide, such as tetrabutylammonium bromide or iodide. A carboxylic acid can be converted in situ into a mixed anhydride by reaction with isobutyl chloroformate. Examples of bis(acyl chlorides) include succinyl dichloride, glutaryl dichloride, adipoyl dichloride, suberoyl dichloride, sebacoyl dichloride, isophthaloyl dichloride, terephthaloyl dichloride, 4,4′-oxybisbenzoyl chloride, 3,3′-methylenebisbenzoyl chloride, bicyclo[2.2.1]hept-5-ene-2,3-dicarbonyl dichloride, tetra-O-acetylgalactaroyl dichloride, 3,6,9-trioxaundecane-1,11-dioic chloride, 3,6-dioxaoctane-1,8-dioic chloride, 3-oxapentane-1,5-dioic chloride, and polyethylene glycol bis(chloroformylmethyl)ether. Examples of bis(acyl bromides) include succinyl dibromide, glutaryl dibromide, adipoyl dibromide, suberoyl dibromide, sebacoyl dibromide, isophthaloyl dibromide, terephthaloyl dibromide, 4,4′-oxybisbenzoyl bromide, 3,3′-methylenebisbenzoyl bromide, and bicyclo[2.2.1]hept-5-ene-2,3-dicarbonyl dibromide. Examples of bis(acyl iodides) include succinyl diiodide, glutaryl diiodide, adipoyl diiodide, suberoyl diiodide, sebacoyl dibromide, isophthaloyl diiodide, terephthaloyl diiodide, 4,4′-oxybisbenzoyl iodide, 3,3′-methylenebisbenzoyl iodide, and bicyclo[2.2.1]hept-5-ene-2,3-dicarbonyl diiodide. Examples of alkyl dichlorides include 1,4-dichlorobutane, 1,5-dichloropentane, 1,6-dichlorohexane, 1,8-dichlorooctane, 1,8-dichloro-3,6-dioxaoctane, 1,11-dichloro-3,6,9-trioxaundecane, α,α′-dichloro-p-xylene, and α,α′-dichloro-m-xylene. Examples of alkyl dibromides include 1,4-dibromobutane, 1,5-dibromopentane, 1,6-dibromohexane, 1,8-dibromooctane, 1,8-dibromo-3,6-dioxaoctane, 1,11-dibromo-3,6,9-trioxaundecane, and α,α′-dibromo-p-xylene. Examples of alkyl diiodides include 1,4-diiodobutane, 1,5-diiodopentane, 1,6-diiodohexane, 1,8-diiodooctane, 1,8-diiodo-3,6-dioxaoctane, 1,11-diiodo-3,6,9-trioxaundecane, and α,α′-diiodo-p-xylene. Examples of sulfonate esters include methanesulfonate, p-toluenesulfonate, trifluoromethanesulfonate and 2,2,2-trifluoroethylsulfonate esters. Examples of alkyl bis(sulfonate esters) include 1,3-bis(p-toluenesulfonyloxy)propane, 1,4-bis(2,2,2-trifluoroethanesulfonyloxy)butane, 1,5-bis(trifluoromethanesulfonyloxy)pentane, 1,6-bis(methanesulfonyloxy)hexane, 1,8-bis(p-toluenesulfonyloxy)octane, 1,10-bis(trifluoromethanesulfonyloxy)decane, 1,12-bis(methanesulfonyloxy)dodecane, 1,14-bis(p-toluenesulfonyloxy)tetradecane, 1,16-bis(methanesulfonyloxy)hexadecane, 1,4-bis(methanesulfonyloxymethyl)benzene, 1,3-bis(p-toluenesulfonyloxymethyl)benzene, mannitol 1,6-dimethanesulfonate, 1,14-bis(trifluoromethanesulfonyloxy)-3,6,9,12-tetraoxatetradecane, 1,11-bis(trifluoromethanesulfonyloxy)-3,6,9-trioxaundecane, 1,8-bis(trifluoromethanesulfonyloxy)-3,6-dioxaoctane, polyethyleneglycolbis(methanesulfonate), and polyethylene glycol bis(p-toluenesulfonate). Examples of diepoxyalkanes include 1,3-diglycidyloxybenzene, 1,4-diglycidyloxybenzene, 1,2-diglycidyloxyethane, 1,4-bis(glycidyloxy)butane, 1,6-bis(glycidyloxy)hexane, 1,2:15,16-diepoxy-4,7,10,13-tetraoxahexadecane, 1,2:12,13-diepoxy-4,7,10-trioxatridecane, bis(4-glycidyloxyphenyl)methane, 1,2:7,8-diepoxyoctane, and 4,4′-diglycidyloxybiphenyl. Examples of diisocyanates include 1,4-diisocyanatobenzene, 1,3-diisocyanatobenzene, 2,6-diisocyanatotoluene, 4,4′-bis(isocyanatophenyl)methane, 1,4-diisocyanatocyclohexane, 1,3-diisocyanatocyclohexane, 1,3-bis(isocyanatomethyl)benzene, isophorone diisocyanate, 4,4′-diisocyanatodicyclohexylmethane, tetramethylene diisocyanate, hexamethylene diisocyanate, bis(2-isocyanatoethyl)ether, and 1,8-diisocyanato-3,6-dioxaoctane.

In some embodiments, R¹ and R² can be independently —CH₂—CH₂—, —CH₂(CH₂)₄CH₂—, Formula II, Formula III, or Formula IV, shown below,

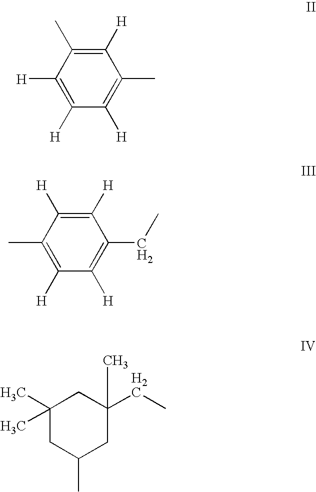

wherein the open valences indicate where R¹ and R² are attached to the nitrogens in Formula I and wherein, when R¹ or R² is Formula IV, either open valence can be attached to the terminal, primary amino (NH₂) group of Formula I.

In some embodiments, R³ and R⁶ can be independently hydrogen or methyl, and R⁴ and R⁵ are independently selected from —CH₂—, —CH(CH₃)—, —CH₂(CH₂)₂CH₂CH(NH₂)—, or —CH₂(CH₂)₂CH₂CH[NHC(═O)O-tert-butyl]-.

In some embodiments, R¹⁰ and R¹¹ can be independently —CH₂CH₂—, —CH₂CH₂CH₂—, or Formula XXIII, shown below.

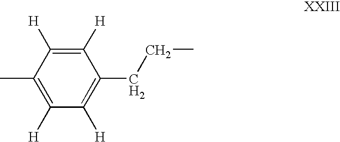

In the above formulae, the open valences indicate attachment to compounds having Formula I, V or XXII. Where the groups are unsymmetrical, both orientations are intended, unless the resulting chemical structure is unstable.

In some embodiments, the compounds of Formula I, V or XXII are prepared in situ in the method described above. The compounds can be prepared in-situ by the process comprising contacting at least one reactive intermediate with a compound of Formula VIII, IX, or X, shown below

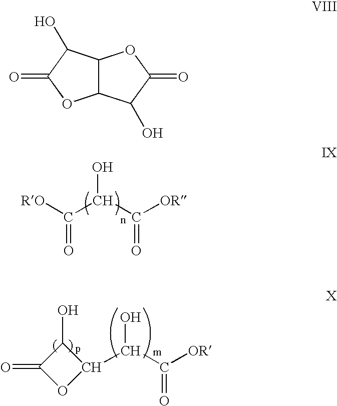

wherein R′ and R″ are independently a 1 to 6 carbon alkyl group, n=1-6, m=0-4, and p=1-4;

wherein the reactive intermediate is selected from one or more or a diamine of the formula NH₂—R⁷—NH₂, an amino acid or amino acid ester of the formula (R⁸OOC)—R⁹—NH₂ or an aminoalcohol of the Formula HO—R¹⁰—NH₂, or salts thereof, wherein R⁷, R⁹, and R¹⁰ are optionally substituted hydrocarbylene groups, wherein the hydrocarbylene groups are aliphatic or aromatic, linear, branched, or cyclic, and wherein the hydrocarbylene groups optionally contain —O— linkages, and wherein R⁸ is independently hydrogen, optionally substituted aryl or optionally substituted alkyl.

In some embodiments, n is equal to 4 or m is equal to 1 and p is equal to 2.

In some embodiments, R⁷, R⁹, and R¹⁰ can be an alkylene polyoxaalkylene, or arylene group, linear, branched, or cyclic, wherein the alkylene polyoxaalkylene, or arylene group is optionally substituted with NH₂ or alkyl.

In some embodiments, the diamine is H₂NCH₂CH₂NH₂, H₂NCH₂(CH₂)₄CH₂NH₂, Formula XI, Formula XII, or Formula XIII, shown below.

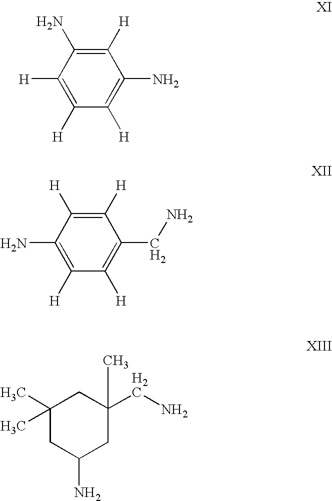

In some embodiments, the amino acid or amino acid ester is H₂NCH₂C(═O)OCH₃, H₂NCH(CH₃)C(═O)OCH₃, H₂N(CH₂)₄CH(NH₂)C(═O)OCH₃, H₂NCH(CH₃)C(═O)OH, H₂N(CH₂)₄CH(NH₂)C(═O)OH, or Formula XX, shown below.

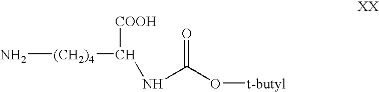

In yet other embodiments, the aminoalcohol is HO—(CH₂)₂—NH₂, HO—(CH₂)₃—NH₂, or 4-(2-aminoethyl)-phenol.

The methods of the instant invention will vary depending on compounds and solvents selected, but can be carried out, for example at a temperature of 20° C. to 130° C. for a time of 1 hour to 3 days. It can be done in the presence of a suitable solvent. Suitable solvents include, for example, water, dimethylformamide, dimethylformamide LiCl, dimethylacetamide, dimethylacetamide LiCl, ethanol and methanol. A “suitable solvent” is a solvent that dissolves or disperses the reactants sufficiently to allow them to react within 3 days at a temperature equal to or less than about 130° C. and is not detrimental to reactant or product.

In some embodiments, the monomer contains functional groups selected from halide, acid chloride, isocyanate, or epoxide.

The invention is also directed to polymers prepared by the methods disclosed herein.

Another aspect of the invention is a method to crosslink a polymer comprising contacting a suitable polymer with one or more crosslinking agents of Formula I, V or XXII:

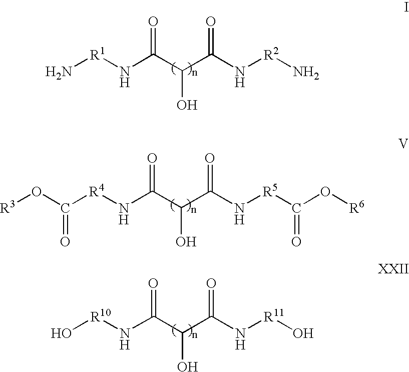

wherein n=1-6, R¹, R², R⁴, R⁵, R¹⁰, and R¹¹ are independently optionally substituted hydrocarbylene groups, wherein the hydrocarbylene groups are aliphatic or aromatic, linear, branched, or cyclic, and wherein the hydrocarbylene groups optionally contain —O— linkages, and R³ and R⁶ are independently hydrogen, optionally substituted aryl or optionally substituted alkyl.

In some embodiments, n is equal to 4.

R¹, R², R⁴, R⁵, R¹⁰, and R¹¹ can be independently alkylene, polyoxaalkylene, or arylene groups, linear or branched, wherein the alkylene, polyoxaalkylene, or arylene groups are optionally substituted with NH₂ or alkyl. When R¹, R², R¹⁰, or R¹¹ is alkylene, it can have from 2 to 20 carbon atoms, preferably from 2 to 8. When R⁴ or R⁵ is alkylene, it can have from 1 to 12 carbon atoms, preferably from 1 to 6. In some embodiments, R¹ and R², R⁴ and R⁵, R³ and R⁶, or R¹⁰ and R¹¹ can be the same.

By “polyoxaalkylene” is meant linear or branched alkyl groups linked by ether linkages. Polyoxaalkylene can contain 2 carbons up to polymeric length units. Examples of polymeric polyoxaalkylenes suitable for the present inventions include polyethyleneglycols, polypropylene glycols, and polytetramethylene glycols such as those based on Terathane® polytetramethyleneetherglycol (E. I. DuPont de Nemours, Wilmington, Del.).

In some embodiments, R¹, R², R⁴, R⁵, R¹⁰, and/or R¹¹ can be an alkylene, polyoxaalkylene, heteroarylene, or arylene group, linear or branched, wherein the alkylene, polyoxaalkylene, heteroarylene or arylene group is optionally substituted with NH₂, aryl including heteroaryl, or alkyl. In some embodiments, n is 4. When R¹, R², R¹⁰, or R¹¹ is alkylene, it can have from 2 to 20 carbon atoms, preferably from 2 to 8. When R⁴ or R⁵ is alkylene, it can have from 1 to 12 carbon atoms, preferably from 1 to 6. Also, “arylene” is intended to include arenedialkylene, e.g.,

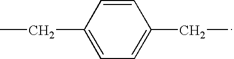

When R¹, R², R⁴, R⁵, R¹⁰, and/or R¹¹ is arylene, it can have from 2 to 12 carbon atoms, preferably 4 to 6. For example, when R¹, R², R⁴, R⁵, R¹⁰, and/or R¹¹ has two carbon atoms, it can be a heteroarylene, e.g., a triazole ring. When R¹, R², R⁴, R⁵, R¹⁰, and/or R¹¹ has 12 carbon atoms, it can be, for example, a biphenyl. When R¹, R², R⁴, R⁵, R¹⁰, and/or R¹ has 4 carbon atoms, examples are furan or pyrrole rings.

When R¹, R², R⁴, R⁵, R¹⁰, and/or R¹¹ is polyoxaalkylene, it can have from 1 to 50 repeat units, preferably from 1 to 10. The total number of carbons depends on the number of carbons in the repeat unit.

In some embodiments, R¹ and R² can be independently —CH₂—CH₂—, —CH₂(CH₂)₄CH₂—, Formula II, Formula II, or Formula IV, shown below,

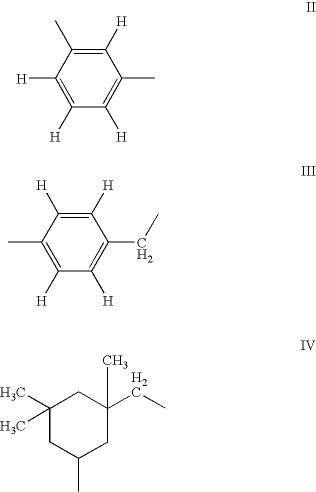

wherein the open valences indicate where R¹ and R² are attached to the nitrogens in Formula I and wherein, when R¹ or R² is Formula IV, either open valence can be attached to the terminal, primary amino (NH₂) group of Formula I.

In some embodiments, R³ and R⁶ can be independently hydrogen or methyl, and R⁴ and R⁵ are independently selected from —CH₂—, —CH(CH₃)—, —CH₂(CH₂)₂CH₂CH(NH₂)—, or —CH₂(CH₂)₂CH₂CH[NHC(═O)O-tert-butyl]-.

In some embodiments, R¹⁰ and R¹¹ can be independently —CH₂CH₂—, —CH₂CH₂CH₂—, or Formula XXIII, shown below.

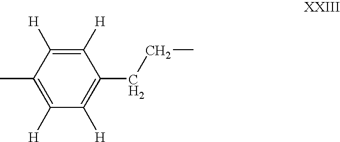

In the above formulae, the open valences indicate attachment to compounds of Formula I, V or XXII. Where the groups are unsymmetrical, both orientations are intended, unless the resulting chemical structure is unstable.

Suitable polymers are those that have functional groups that react at a temperature less than or equal to about 130° C. with the primary amine groups of Formula I to form carbon-nitrogen bonds, the ester groups of Formula V to form bonds to the carbonyl carbon atom, or the hydroxyl groups of Formula XXII to form carbon-oxygen bonds. In some embodiments, the polymer is selected from polyallylamine, polyethyleneimine, polylysine, chitosan, and derivatives and salts thereof; polyether amines such as hexakis(aminoethyl)sorbitol ethoxylate (P2809-6EONH2, Polymer Source, Inc., Montreal, Quebec, Canada) and Jeffamine T 403 (Huntsman, Houston, Tex.) and salts thereof; polyether portions can be poly(ethylene glycol), poly(propylene glycol), poly(1,3-propanediol), poly(tetrahydrofuran) (Terathane®), or copolymers, wherein the endgroups can be 2-aminoethyl, 2-aminopropyl, 3-aminopropyl, or 4-aminobutyl; aminoethyl starch, aminopropyl starch, aminoethyl cellulose, aminopropyl cellulose, aminoethyl dextran, aminopropyl dextran, aminoethyl inulin, aminopropyl inulin, derivatives and salts thereof; aminoethyl poly(vinyl alcohol) and aminopropyl poly(vinyl alcohol), derivatives and salts thereof; poly(vinyl amine), copolymers, derivatives and salts thereof; poly(alkyl acrylate), poly(alkyl methacrylate); and poly(acryloyl chloride), poly(methacryloyl chloride).

The methods disclosed herein for crosslinking polymers will vary depending on compounds and solvents selected, but can be carried out, for example, at a temperature of 20° C. to 130° C. for a time of 1 hour to 3 days. It can be done in the presence of a suitable solvent. A suitable solvent can be water, dimethylformamide, dimethylformamide LiCl, dimethylacetamide, dimethylacetamide LiCl, ethanol or methanol.

In some embodiments, the crosslinking agent is a compound of Formula I or Formula V.

Another aspect of the invention is a crosslinked polymer made by the methods described above, in which a suitable polymer is contacted with one or more crosslinking agents of Formula I, V or XXII.

### EXAMPLES

The present invention is illustrated by the following Examples. It should be understood that these Examples, while illustrating some preferred embodiments of the invention, are given by way of illustration only. From the above discussion and these Examples, one skilled in the art can ascertain the preferred embodiments of this invention, and without departing from the spirit and scope thereof, can make various changes and modifications of the invention to adapt it to various uses and conditions.

In Situ Generation of Diaminoaldaramides and Use as Monomers

### Example 1

### DMG+HMD+α,α′-Dichloro-p-xylene

Into a 250-mL 3-neck round-bottom flask equipped with a heating mantle, reflux condenser, nitrogen inlet, and overhead stirrer was added 13 mL of dimethylformamide (DMF), 13 mL of methanol, and 4.64 g (0.040 mol) of hexamethylenediamine. The mixture was stirred at room temperature until the diamine dissolved. At this point, 3.81 g (0.016 mol) of DMG was added to the solution and the resulting mixture was heated to reflux. The mixture was then held at reflux for 20 minutes, after which time the heating mantle was removed and 4.20 g (0.024 mol) of α,α′-dichloro-p-xylene were added, followed immediately by the addition of 3.5 g (0.033 mol) of sodium carbonate. The mixture was then heated to reflux for approximately 2.5 hours until a thick gel formed. The gel was then maintained at a low heat level for an additional 21 hours. The resulting gel was then removed from the flask and washed 3 times with 100 mL of ethanol, followed by aqueous ammonia, water, 22% (w/w) HCl, water, and ethanol. The gel was then dried in a vacuum oven set at 80° C. to 100° C. to yield 4.80 g (41.3%) of a white, granular hydrogel polymer. T_{dec} (TGA) 260° C. (onset); swell 8.62 g H₂O/g polymer; θᵢₙₕ (HFIP) insoluble.

While the preceding Example used DMG and α,α′-dichloro-p-xylene in a 40:60 mole ratio, polymers employing different ratios were prepared similarly (Table 1).

TABLE 1
Polymers Prepared From DMG and α,α′-Dichloro-p-xylene

| | mole ratio HMD:α,α′-dichloro-p-xylene | | | | |
|---|---|---|---|---|---|
| | 50:50 | 40:60 | 30:70 | 20:80 | 10:90 |
| yield, % | 38 | 41 | 44 | 58 | — |
| ηᵢₙₕ (HFIP) | insoluble | insoluble | insoluble | insoluble | insoluble |
| swell, | 4.04 | 8.62 | 3.12 | 5.88 | 7.04 |
| g_{H}_{₂}_{O}/g | | | | | |
| T_{g}, ° C. | — | — | — | — | 49.52 |
| Tₘ, ° C. | 158.33 | — | — | — | — |
| ΔH, J/g | 1.882 | — | — | — | — |
| T_{dec}, ° C. | 260 | 260 | 260 | 275 | 260 |

### Example 2

### DMG+HMD+1,2:7,8-Diepoxyoctane

Into a 250-mL 3-neck round-bottom flask equipped with a heating mantle, reflux condenser, nitrogen inlet, and overhead stirrer were added 13 mL of DMF, 13 mL of methanol, and 4.64 g (0.040 mol) of hexamethylenediamine. The resulting mixture was stirred at room temperature for about 10 minutes until a homogeneous solution resulted. At this point, 4.76 g (0.020 mol) of DMG were added to the solution, and the mixture was heated at reflux for 20 minutes. The heat source was removed, and 2.84 g (0.020 mol) of 1,2:7,8-diepoxyoctane were added to the mixture. Heat was applied once again with stirring to attain reflux. Reflux was maintained for approximately 4 hours, until gel formation occurred. At this point heating was stopped, and the gel was left to stir for an additional 19 hours at room temperature. The resulting gel was removed from the flask and washed 3 times with 100 mL of ethanol, followed by aqueous ammonia, water, 22% HCl, and water. The gel was then dried in a vacuum oven at about 40° C. to yield 10.09 g (92.1%) of a white granular hydrogel. T_{dec} (TGA) 260° C. (onset); swell 1.75 g H₂O/g polymer.

While the preceding Example used DMG and 1,2:7,8-diepoxyoctane in a 50:50 mole ratio, polymers employing different ratios were prepared similarly (Table 2).

TABLE 2
Polymers Prepared Using DMG and 1,2:7,8-diepoxyoctane

| | mole ratio HMD:1,2:7,8-diepoxyoctane | | | | |
|---|---|---|---|---|---|
| | 50:50 | 40:60 | 30:70 | 20:80 | 10:90 |
| yield, % | 92 | 93 | 99 | 90 | 85 |
| ηᵢₙₕ (HFIP) | insoluble | insoluble | insoluble | insoluble | insoluble |
| swell, | 1.75 | 0.33 | 1.82 | 1.73 | 7.31 |
| g_{H}_{₂}_{O}/g | | | | | |
| T_{g}, ° C. | — | 48.4 | — | — | — |
| Tₘ, ° C. | — | 182 | 184.2 | 185.8 | 139.3 |
| ΔH, J/g | — | 57.74 | 62.57 | 48.1 | 3.026 |
| T_{dec}, ° C. | 280 | 180 | 200 | 210 | 270 |

### Example 3

### DMG+HMD+MDI

Into a 250-mL 3-neck round-bottom flask equipped with a heating mantle, reflux condenser, nitrogen inlet, and overhead stirrer were added 26 mL of DMAC and 4.64 g (0.040 mol) of hexamethylenediamine. The resulting mixture was stirred at room temperature for 5 minutes until a homogeneous solution resulted. At this point, 4.76 g (0.020 mol) of DMG were added to the solution, and the mixture was heated at reflux for 25 minutes. The heat source was removed, and 5.0 g (0.020 mol) of 4,4′-methylenebis(phenyl isocyanate) were added to the mixture. Heat was applied once again with stirring to attain reflux. Reflux was maintained for 9 hours, until gel formation occurred. At this point heating was stopped, and the gel was left to stir for an additional 14 hours at room temperature. The resulting gel was removed from the flask, washed 3 times with 100 mL of ethanol, followed by aqueous ammonia, water, 22% HCl, and water. The gel was then dried in a vacuum oven at 40° C. to yield 13.2 g (100%). T_{g} 115.58° C.; Tₘ 205.7° C. (ΔH 9.063 J/g); T_{dec} (TGA) 225° C. (onset); θᵢₙₕ (HFIP) insoluble; swell 0.33 g H₂O/g polymer.

While the preceding Example used DMG and 4,4′-methylenebis(phenyl isocyanate) in a 50:50 mole ratio, polymers employing different ratios were prepared similarly (Table 3).

TABLE 3
Polymers Prepared From DMG and 4,4′-Methylenebis(phenyl
isocyanate)

| | mole ratio HMD:4,4′-methylenebis(phenyl isocyanate) | | | | |
|---|---|---|---|---|---|
| | 50:50 | 40:60 | 30:70 | 20:80 | 10:90 |
| yield, % | 38 | 41 | 44 | 58 | — |
| ηᵢₙₕ (HFIP) | insoluble | insoluble | insoluble | insoluble | insoluble |
| swell, | 4.04 | 8.62 | 3.12 | 5.88 | 7.04 |
| g_{H}_{₂}_{O}/g_{polymer} | | | | | |
| T_{g}, ° C. | 116 | 112 | 110 | 114 | 121 |
| Tₘ₁, ° C. | 205.7 | 232.7 | 219.9 | 224.2 | 218 |
| ΔH, J/g | 9.063 | 49.94 | 17.98 | 15.93 | 6.689 |
| Tₘ₂, ° C. | 231.46 | — | 229.71 | 231.73 | 228.84 |
| ΔH, J/g | 65.22 | — | 10.75 | 4.937 | 1.029 |
| T_{c}, ° C. | — | 175.2 | — | — | — |
| ΔH, J/g | — | 0.3976 | — | — | — |
| T_{dec}, ° C. | 225 | 225 | 225 | 230 | 230 |

### Example 4

### DMG+Ethylenediamine+MDI-Capped Terathane®

Into a 250-mL 3-neck round-bottom flask equipped with a heating mantle, reflux condenser, nitrogen inlet, and overhead stirrer were added 25 mL of DMAC and 0.590 g (9.82 mmol) of ethylenediamine. The mixture was stirred at room temperature until the diamine dissolved, and DMG (0.750 g, 3.15 mmol) was added. Heating the mixture at reflux for 20 minutes gave a milky suspension. To this mixture, held at 70° C. with stirring, was added a solution of 10.0 g (6.67 mmol) of Terathane® end-capped with 4,4′-methylenebis(phenyl isocyanate), MW 1,500, in 10 mL of dry DMAC. The temperature of the stirred mixture was raised to 90° C. and held there for 20 hours. A small amount of the resulting solution was spread on a glass plate with a blade applicator to form a film, and the plate was placed in a vacuum oven at 80° C. to remove the solvent. The resulting film (0.25 inch×2 inches) had the following properties: thickness 3.40 mil; stress at break 1,332 psi; strain at break 305.54%; initial modulus 4,054 psi. The remaining reaction solution was poured into water, and the resulting precipitate was collected by filtration and dried in a vacuum oven at 80° C. to give 6.47 g of a rubbery polymer: T_{g} −54.83° C.; T_{c} −9.93° C. (ΔH 8.012 J/g); Tₘ 13.99° C. (ΔH 9.321 J/g); 258.35° C. (ΔH 15.49 J/g); T_{dec} (TGA) 240° C. (onset); ƒᵢₙₕ (m-cresol) 0.721.

While the preceding Example used DMG and MDI-capped Terathane® in a 32:68 mole ratio, polymers employing different ratios were prepared similarly (Table 4).

TABLE 4
Polymers Prepared From DMG and MDI-Capped Terathane ®

| | mole ratio HMD:MDI-capped Terathane ® | | | | | |
|---|---|---|---|---|---|---|
| | 75:25 | 50:50 | 40:60 | 32:68 | 20:80 | 10:90 |
| yield, % | — | — | — | — | — | — |
| ηᵢₙₕ (m-cresol) | insoluble | 0.398 | 0.819 | 0.721 | 1.023 | 0.585 |
| swell, g_{H}_{₂}_{O}/g_{polymer} | — | — | — | — | — | — |
| T_{g}, ° C. | −52.2 | — | −59.5 | −54.83 | −57.6 | −56.3 |
| Tₘ₁, ° C. | 13.67 | 128.3 | 16.29 | 13.99 | 14.14 | 16.04 |
| [symbol] ΔH, J/g | 1.895 | 0.2161 | 13.4 | 9.321 | 9.985 | 0.9976 |
| Tₘ₂, ° C. | 156.2 | — | 192.8 | 258.35 | 212.61 | 206.44 |
| [symbol] ΔH, J/g | 163.5 | — | 2.397 | 15.49 | 0.5153 | 3.784 |
| Tₘ₃, ° C. | — | — | — | — | — | 275.25 |
| [symbol] ΔH, J/g | — | — | — | — | — | 28.61 |
| T_{c}, ° C. | −14.4 | 134.7 | −19.06 | −9.93 | −14.51 | — |
| [symbol] ΔH, J/g | 0.8809 | 0.7546 | 15.04 | 8.012 | 9.389 | — |
| T_{dec}, ° C. | 190 | 240 | 225 | 240 | 235 | 235 |
| film thickness, mil | 1.583 | 3.067 | 6.225 | 3.400 | 3.600 | 4.550 |
| initial modulus, psi | 13,340 | 4,230 | 1,150 | 4,054 | 913 | 4,681 |
| stress @ yield, psi | 788 | 396 | 416 | 1,350 | 449 | 953 |
| stress @ max, psi | 793 | 400 | 423 | 1,353 | 453 | 961 |
| stress @ break, psi | 655 | 368 | 403 | 1,332 | 421 | 930 |
| stress @ 10%, psi | 644 | 305 | 116 | 419 | 91 | 413 |
| strain @ yield, % | 34.512 | 20.08 | 282.97 | 300.08 | 440.53 | 100.51 |
| strain @ max, % | 51.82 | 19.58 | 316.74 | 301.08 | 451.93 | 113.19 |
| strain @ break, % | 118.37 | 20.88 | 357.64 | 305.54 | 474.33 | 125.07 |

### Example 5

### DMG+Ethylenediamine+Sebacoyl Chloride

Into a 250-mL 3-neck round-bottom flask equipped with a heating mantle, reflux condenser, nitrogen inlet, and overhead stirrer were added 35 mL of a 3.8% solution of lithium chloride in DMAC, 1.20 g (20.0 mmol) of ethylenediamine, and 2.38 g (10.0 mmol) of DMG. The mixture was heated at 50° C. for 30 minutes. External heating was discontinued, and sebacoyl chloride (4.78 g, 20.0 mmol) was added dropwise over 13 minutes, during which the temperature of the reaction rose from 35° C. to 44° C. Calcium hydroxide (1.5 g, 20 mmol) was added, external heating was resumed, and the mixture was stirred at 50° C. for 19.5 hours. The reaction was poured into THF, and the resulting precipitate was collected by filtration and dried in a vacuum oven to give 4.17 g of product (66% yield): T_{g} 49.06° C.; T_{dec} (TGA) 200° C. (onset); ηᵢₙₕ (HFIP) insoluble.

Use of Isolated α,ω-Difunctional Aldaramides as Monomers

### Example 6

### DMG+m-Phenylenediamine+Isophthaloyl Chloride

Into a 250-mL 3-neck round-bottom flask equipped with a thermometer and overhead stirrer were added 50 mL of a 3.8% solution of lithium chloride in DMAC, 5.13 g (47.5 mmol) of m-phenylenediamine, and 0.98 g (2.5 mmol) of N¹,N⁶-bis(3-aminophenyl)galactaramide. The stirred mixture was heated gently to dissolve all ingredients and then cooled to about 0° C. using an ice bath. Isophthaloyl chloride (10.15 g, 50.0 mmol) was added. The reaction temperature climbed quickly to about 50° C. and then cooled to about 10° C. within 20 minutes. The ice bath was removed, and the reaction was allowed to warm to room temperature over 2 hours. Calcium hydroxide (3.7 g, 50 mmol) was added, and the temperature climbed quickly to about 50° C. and then cooled to room temperature within 2 hours. The mixture was stirred at room temperature for an additional 16 hours and poured into water. The resulting precipitate was collected by filtration and dried in a vacuum oven at 60° C. to give 7.60 g of a powdery solid: Tₘ 108.9° C. (ΔH 0.2745 J/g); T_{g} 260.89° C.; ηᵢₙₕ (4% LiCl in DMAC) 0.345.

### Example 7

### DMG+4-Aminobenzylamine+Isophthaloyl Chloride

In the same way as in the preceding Example, isophthaloyl chloride was reacted with a 95:5 mole ratio of m-phenylenediamine and N¹,N⁶-bis(4-aminobenzyl)galactaramide: 77% yield; T_{g} 259° C.; T_{dec} (TGA) 250° C. (onset); ηᵢₙₕ (4% LiCl in DMAC) 0.316.

### Example 8

### GDL+4-Aminobenzylamine+Isophthaloyl Chloride

In the same way as in the preceding Example, isophthaloyl chloride was reacted with a 95:5 mole ratio of m-phenylenediamine and N¹,N⁶-bis(4-aminobenzyl)-D-glucaramide: 74% yield; T_{g} 252° C.; T_{dec} (TGA) 250° C. (onset); ηᵢₙₕ (4% LiCl in DMAC) 0.330.

Use of Isolated α,ω-Difunctional Aldaramides as Crosslinkers

### Example 9

### N¹,N⁶-Bis(2-aminoethyl)-D-glucaramide+poly(methacryloyl chloride)

Into a 250-mL 3-neck round-bottom flask equipped with a heating mantle, reflux condenser, nitrogen inlet, and overhead stirrer was added 25 mL of dioxane containing 6.25 g (0.598 equivalent) of poly(methacryloyl chloride) (Polysciences, Inc., Warrington, Pa.). To this solution was added 3.50 g (15.0 mmol) of N¹,N⁶-bis(2-aminoethyl)-D-glucaramide. The heterogeneous mixture was stirred at 50° C. for 21 hours. It was then poured into THF and filtered, and the solid collected was washed 3 times with THF to give 2.65 g (27%) of a light tan solid: T_{g1} 49.67° C.; T_{g2} 64.14° C.; T_{dec} 175° C. (onset); ηᵢₙₕ (HFIP) insoluble.

### Example 10

### N¹,N⁶-Bis(methoxycarbonylmethyl)-D-glucaramide+PAH

In a dry box, triethylamine (11.7 mL, 84.0 mmol) was added to a solution of polyallylamine hydrochloride (MW ca. 60,000, 6.55 g, 70.0 mmol) in 270 mL of methanol in a 500-mL round-bottom flask equipped with a magnetic stirbar. To the resulting solution was added a slurry of N¹,N⁶-bis(methoxycarbonylmethyl)-D-glucaramide (0.25 g, 0.69 mmol) in methanol (20 mL). The resulting solution was stirred at ambient temperature for four days. The reaction solvent was removed under vacuum, and the oily solid was washed repeatedly with methanol (180 mL). Addition of pentane (50 mL) to a slurry of the product in 20 mL of methanol gave a solid that was collected by filtration and dried in vacuum to give 2.39 g (57% yield) of a solid that exhibited a swell ratio (after 29 minutes of suction) of 62.8. When the swell test was repeated, allowing 16 hours for the gel to swell followed by 34 minutes of suction, the swell ratio was 118.9. After 23 hours' exposure to ambient atmosphere, the sample retained 108.6 times its own weight in water.

In a similar way, N¹,N⁶-bis(methoxycarbonylmethyl)-D-glucaramide (0.14 g, 0.41 mmol) in water (1.5 mL) was added to a solution of polyallylamine hydrochloride (MW Ca. 60,000, 1.01 g, 10.8 mmol) and sodium hydroxide (0.033 g, 0.83 mmol) in water (3 mL), and the mixture was stirred at ambient temperature for 45 hours. The solvent was evaporated under reduced pressure, and sodium chloride was removed from the residue by washing with methanol (125 mL) to give a white solid (0.98 g, 89% yield) that exhibited a swell ratio (after 5 minutes of dynamic suction and 45 minutes of static suction) of 105.8. After 2 days' exposure to ambient atmosphere, the sample retained 96.5 times its own weight in water. When the swell test was repeated with the same sample, allowing 4.5 hours for the gel to swell followed by 5 hours of dynamic suction and 14 hours of static suction, the swell ratio was 197.6. After 6 days' exposure to ambient atmosphere, the sample retained 167.8 times its own weight in water.

### Example 11

### N¹′,N⁶-Bis(methoxycarbonylmethyl)-D-glucaramide+PEI

Polyethylenimine (Mₙ=ca. 10,000, M_{w}=ca. 25,000, Aldrich 408727, 0.67 g, 15.6 mmol) was weighed into a 20-mL scintillation vial equipped with a magnetic stirbar, and water (2.5 mL) was added. Concentrated hydrochloric acid (0.65 mL) was added dropwise to the solution followed by solid N¹,N⁶-bis(methoxycarbonylmethyl)-D-glucaramide (0.14 g, 0.39 mmol) and water (1 mL). The reaction stirred for 5 days at ambient temperature. The solvent was then removed under vacuum, and the solid was vacuum-dried to give a colorless solid that exhibited a swell ratio (after 50 minutes of dynamic suction and 15 minutes of static suction) of 17.6. When the swell test was repeated with the same sample, allowing 15 hours for the gel to swell followed by 2.25 hours of suction, the swell ratio was 25.5. After five days' exposure to ambient atmosphere, the sample retained 22.8 times its own weight in water.

What is claimed is:

1. A method of preparing a polymer comprising: contacting one or more suitable monomers with a compound of Formula I, V or XXII: 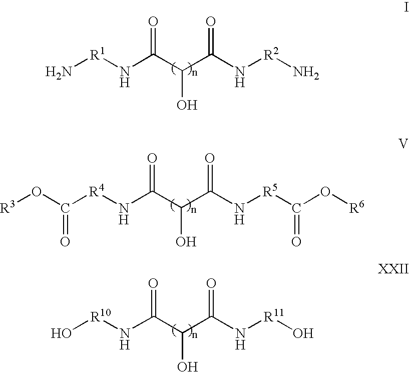
wherein n=1-6, R¹, R², R⁴, R⁵, R¹⁰, and R¹¹ are independently optionally substituted hydrocarbylene groups, wherein the hydrocarbylene groups are aliphatic or aromatic, linear, branched, or cyclic, and wherein the hydrocarbylene groups optionally contain —O— linkages, and R³ and R⁶ are independently hydrogen, optionally substituted aryl or optionally substituted alkyl.

2. The method of claim 1 wherein n=4.

15

3. The method of claim 1 wherein R¹, R², R⁴, R⁵, R¹⁰, and R¹¹ are independently alkylene, polyoxaalkylene, or arylene groups, linear or branched, and wherein the alkylene, polyoxaalkylene, or arylene groups are optionally substituted with NH₂ or alkyl.

4. The method of claim 1 wherein R¹ and R² are the same, R⁴ and R⁵ are the same, R³ and R⁶ are the same, or R¹⁰ and R¹¹ are the same.

5. The method of claim 1 wherein R¹ and R² are independently selected from
—CH₂—, —CH₂—, —CH₂(CH₂)₄CH₂—, and groups of Formula II, Formula III, or Formula IV, 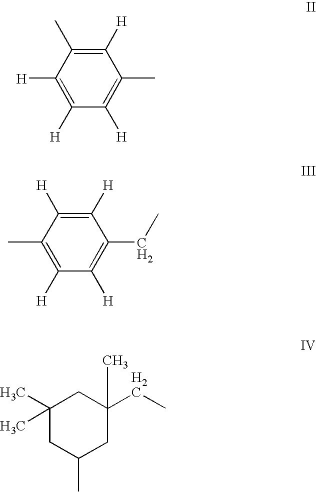
wherein the open valences indicate wherein R¹ and R² are attached to the nitrogens in Formula I and wherein, when R¹ or R² is Formula IV, either of said open valences can be attached to the terminal, primary amino (NH₂) group of Formula I.

6. The method of claim 1 wherein R³ and R⁶ are independently hydrogen or methyl, and R⁴ and R⁵ are independently selected from —CH₂—, —CH(CH₃)—,
—CH₂(CH₂)₂CH₂CH(NH₂)—, and —CH₂(CH₂)₂CH₂CH[NHC(═O)O-tert-butyl]-.

7. The method of claim 1 wherein R¹⁰ and R¹¹ are independently selected from: —CH₂CH₂—, —CH₂CH₂CH₂—, and a group of Formula XXIII.

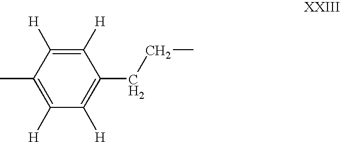

8. The method of claim 1 wherein the compounds of Formula I, V or XXII are prepared in situ.

9. The method of claim 8 wherein the compounds are prepared in situ by a process comprising contacting at least one reactive intermediate with a compound of Formula VIII, IX, or X 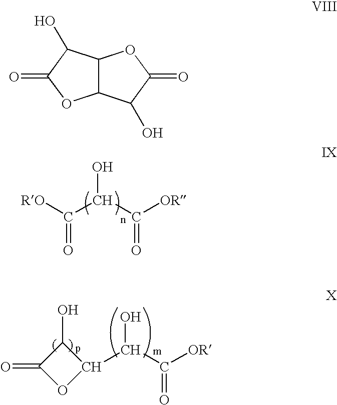
wherein R′ and R″ are independently selected from 1 to 6 carbon alkyl groups, n=1-6, m=0-4, and p=14;
wherein the reactive intermediate is selected from: diamines of formula NH₂—R⁷—NH₂, amino acids and amino acid esters of formula (R⁸OOC)—R⁹—NH₂ and aminoalcohols of formula HO—R¹⁰—NH₂, and salts thereof wherein R⁷, R⁹, and R¹⁰ are optionally substituted hydrocarbylene groups, wherein the hydrocarbylene groups are aliphatic or aromatic, linear, branched, or cyclic, and wherein the hydrocarbylene groups optionally contain —O— linkages, and wherein R⁸ is independently hydrogen, optionally substituted aryl or optionally substituted alkyl.

10. The method of claim 9 wherein n=4 or wherein m is 1 and p is 2.

11. The method of claim 9 wherein R⁷, R⁹, or R¹⁰ is an alkylene polyoxaalkylene, or arylene group, linear, branched, or cyclic, and wherein the alkylene polyoxaalkylene, or arylene group is optionally substituted with NH₂ or alkyl.

12. The method of claim 9 wherein the diamine is H₂NCH₂CH₂NH₂, H₂NCH₂(CH₂)₄CH₂NH₂, Formula XI, Formula XIII, or Formula XIII.

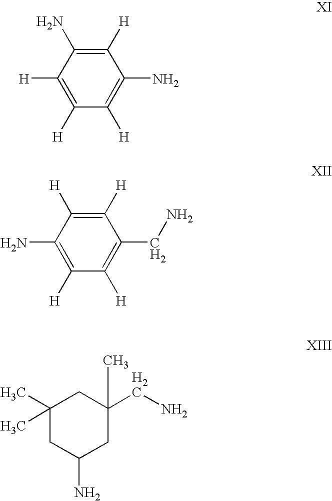

13. The method of claim 9 wherein the amino acid or amino acid ester is H₂NCH₂C(═O)OCH₃, H₂NCH(CH₃)C(═O)OCH₃, H₂N(CH₂)₄CH(NH₂)C(═O)OCH₃, H₂NCH(CH₃)C(═O)OH, H₂N(CH₂)₄CH(NH₂)C(═O)OH, or a group of formula XX

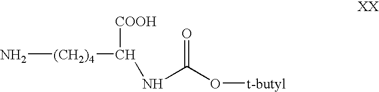

14. The method of claim 9 wherein the aminoalcohol is HO—(CH₂)₂—NH₂, HO—(CH₂)₃—NH₂, or 4-(2-aminoethyl)-phenol.

15. The method of claim 1 wherein the contacting is carried out at a temperature of 20° C. to 130° C. for a time of 1 hour to 3 days.

16. The method of claim 1 wherein the contacting is carried out in the presence of a suitable solvent.

17. The method of claim 16 wherein the suitable solvent is water, dimethylformamide, dimethylformamide LiCl, dimethylacetamide, dimethylacetamide LiCl, ethanol or methanol.

18. The method of claim 1 wherein the monomer contains functional groups selected from halide, acid chloride, isocyanate, and epoxide.

19. The method of claim 1 wherein the polymer is prepared with a compound of Formula I.

20. A polymer made by the method of claim 1.

21. A method for crosslinking a polymer comprising contacting a suitable polymer with one or more compounds of Formula I, V or XXII: 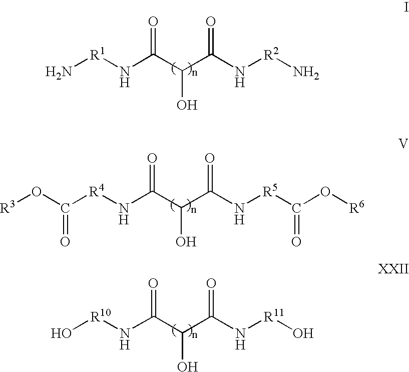
wherein n=1-6, R¹, R², R⁴, R⁵, R¹⁰, and R¹¹ are independently selected from optionally substituted hydrocarbylene groups, wherein the hydrocarbylene groups are aliphatic or aromatic, linear, branched, or cyclic, and wherein the hydrocarbylene groups optionally contain —O— linkages, and R³ and R⁶ are independently hydrogen, optionally substituted aryl or optionally substituted alkyl.

22. The method of claim 21 wherein n=4.

23. The method of claim 21 wherein wherein R¹, R², R⁴, R⁵, R¹⁰, and R¹¹ are independently selected from: alkylene, polyoxaalkylene, and arylene groups, linear, branched, or cyclic, and wherein the alkylene, polyoxaalkylene, or arylene groups are optionally substituted with NH₂ or alkyl.

24. The method of claim 21 wherein R¹ and R² are the same, R⁴ and R⁵ are the same and R³ and R⁶ are the same, or R¹⁰ and R¹¹ are the same.

25. The method of claim 21 wherein R¹ and R² are independently selected from: —CH₂—CH₂—, —CH₂(CH₂)₄CH₂—, and groups of formula II, formula III, or formula IV, 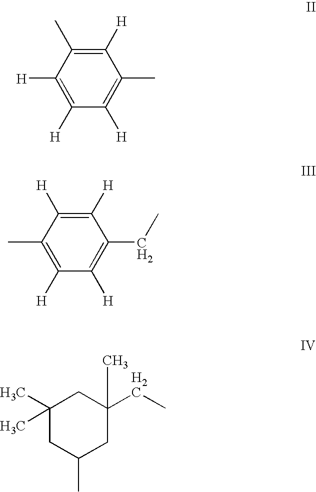
wherein the open valences indicate where R¹ and R² are attached to the nitrogens in Formula I and wherein, when R¹ or R² is Formula IV, either open valence can be attached to the terminal, primary amino (NH₂) group of Formula I.

26. The method of claim 21 wherein R³ and R⁶ are independently hydrogen or methyl, and R⁴ and R⁵ are independently selected from —CH₂—, —CH(CH₃)—,
—CH₂(CH₂)₂CH₂CH(NH₂)—, and —CH₂(CH₂)₂CH₂CH[NHC(═O)O-tert-butyl]-.

27. The method of claim 21 wherein R¹⁰ and R¹¹ are independently —CH₂CH₂—, —CH₂CH₂CH₂—, or a group of formula XXIII.

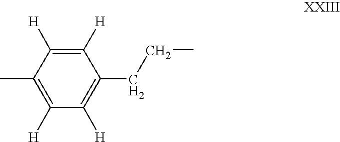

28. The method of claim 21 wherein the contacting is carried out at a temperature of 20° C. to 130° C. for a time of 1 hour to 5 days.

29. The method of claim 21 wherein the contacting is carried out in the presence of a suitable solvent.

30. The method of claim 29 wherein the suitable solvent is water, dimethylformamide, dimethylformamide LiCl, dimethylacetamide, dimethylacetamide LiCl, ethanol or methanol.

31. The method of claim 21 wherein the polymer is selected from: polyallylamine, polyethyleneimine, polylysine, chitosan, polyether amine, aminoethyl starch, aminopropyl starch, aminoethyl cellulose, aminopropyl cellulose, aminoethyl dextran, aminopropyl dextran, aminoethyl inulin, aminopropyl inulin, aminoethyl poly(vinyl alcohol), aminopropyl poly(vinyl alcohol), poly(vinyl amine), poly(alkyl acrylate), poly(alkyl methacrylate), poly(acryloyl chloride), and poly(methacryloyl chloride), and copolymers, derivatives or salts thereof.

32. The method of claim 21 wherein the compound is a compound of Formula I or Formula V.

33. A polymer made by the method of claim 21.
